(19) United States
(12) Patent Application Publication
(10) Pub. No.: US 2006/0084838 A1
(43) Pub. Date: Apr. 20, 2006

(54) Instrument for re-insertion of a gastrostoma tube

(76) Inventor: Norikata Takuma, Kokubunji City (JP)

Correspondence Address: JORDAN AND HAMBURG LLP, 122 EAST 42ND STREET, SUITE 4000, NEW YORK, NY, 10168, US

(21) Appl. No.: 11/242,476

(22) Filed: Oct. 3, 2005

(30) Foreign Application Priority Data
Oct. 4, 2004 (JP) 2004-291208
Mar. 14, 2005 (JP) 2005-70907

(51) Int. Cl. A61B 1/00

(52) U.S. Cl. 600/101

(57) ABSTRACT

A medical instrument provides assistance in reinserting a gastrostoma tube easily. The instrument includes a searching bar and a fistula expander. A main body of the searching bar is a tubular body, a front end of the searching bar presents a hemispherical shape, and a side hole is defined near the front end. The fistula expander includes a funnel-shaped portion divided into two portions at a front end portion of the fistula expander, two grip bars secured to both sides of the funnel-shaped portion respectively, and a spring portion secured to rear ends of the grip bars. One may visually identify, and take photographs of, the location of the front end portion and/or side portion of the searching bar while light from an light source through optical fibers is radiated outwardly via a lens aperture or apertures in the front end and/or front end portion.

### BACKGROUND OF THE INVENTION

1. Field of the Invention

The present invention provides an instrument for re-insertion of a gastrostoma tube which provides assistance in re-inserting a gastrostoma tube so that the gastrostoma tube can be re-inserted easily and safely because the gastrostoma tube is a medical instrument which tends to be pull out when a patient uses the gastrostoma tube. More specifically, the present invention provides: an instrument for re-insertion of a gastrostoma tube comprising a combination of a searching bar and a fistula expander wherein a main body of the searching bar is a tubular body, a front end of the searching bar presents a hemispherical shape, and a side hole is defined near the front end, and wherein the fistula expander comprises a funnel-shaped portion divided into two portions at a front end portion of the fistula expander, two grip bars secured to both sides of the funnel-shaped portion respectively, and a spring portion secured to rear end portions of the grip bars; and an instrument for re-insertion of a gastrostoma tube wherein a lens aperture is defined in the searching bar, and light is transmitted via optical fibers from a light source connected to the optical fibers so that the inside of a stomach can be seen by imvages with an image display, or insertion of the searching bar can be performed while a fistula in close proximity of the front end of the searching bar (i.e. a fistula which will be in the direction to be inserted) can be seen directly by images from the inside of the searching bar.

2. Description of Related Art

Conventionally, nutrition and water have been supplied to a patient who cannot take meals by mouth such as a dementia patient, by providing a shortcut, i.e., gastrostoma (gastric fistula), which connects an abdominal wall and a stomach and inserting a gastrostoma tube into the gastrostoma. In case that the gastrostoma tube being used by such patients being in hospitals or recuperating at home should be pull out for some reason or other, re-insertion of the tube should be performed as soon as possible in compliance with basic medical guidelines.

The reasons why the tube is encouraged to be re-inserted soon is that, after the tube is pulled out, a fistula tends to contract due to tension of muscle which was located around the inserted tube, or a shape of the fistula which penetrates through an abdominal wall to a stomach wall tends to become distorted. Accordingly, it becomes more difficult to re-insert the tube as time goes by.

However, there have not necessarily been an established method of re-insertion in the medical field so far and such a method of re-insertion currently may depends on the individual skills of doctors in charge. Therefore, intra-abdominal mis-insertion may lead to peritonitis.

As for a supporting instrument used for exchanging such a gastrostoma tube, a supporting instrument has been known comprising a guide portion and a handle portion, the guide portion guiding a gastrostoma tube while supporting a fistula, the handle portion which operating the guide portion, as described in Japanese Patent (unexamined) Publication No. 2002-17866, n instrument for supporting a fistula and guiding a gastrostoma tube However, this supporting instrument is inserted along the gastrostoma tube, when the tube is pulled out.

As stated above, it has been desired to re-insert a gastrostoma tube more safely and more reliably when exchanging a gastrostoma tube. If the tube should be pull out, the following problems have to be considered. The problems include:

(i) A fistula may contract due to contraction of muscles of an abdominal wall and a stomach wall.

(ii) Soft tissues such as a stomach wall or an abdominal wall should not be hurt when a re-insertion treatment is performed.

(iii) How the path for a fistula which has contracted to be narrower should be secured after a gastrostoma tube is pulled out.

(iv) How it can be seen whether an instrument is located in a lumen of a stomach as is intended by the manipulator or not.

(v) How a gastrostoma tube is inserted finally.

### SUMMARY OF THE INVENTION

In order to solve the above-mentioned problems, the inventor of the present invention researched thoroughly and found that a path for a fistula after a gastrostoma tube is pulled out can be secured again in a two-stepped way of: firstly securing a path for a fistula by a searching bar; and then, expanding the secured path by a fistula expander, so that a re-insertion instrument for a gastrostoma tube of the present invention can be developed.

A first aspect of the present invention is an instrument for re-insertion of a gastrostoma tube comprising a combination of a searching bar and a fistula expander.

A second aspect of the present invention is an instrument for re-insertion of a gastrostoma tube as described above, wherein a main body of the searching bar is a tubular body, a front end of the searching bar is a hemispherical body, and a side hole is defined near the front end.

A third aspect of the present invention is an instrument for re-insertion of a gastrostoma tube as described above, wherein a main body of the searching bar is a tubular body, and a connecting portion which can be connected to a syringe is provided at a rear end portion of the searching bar.

A fourth aspect of the present invention is an instrument for re-insertion of a gastrostoma tube as described above, wherein the fistula expander comprises a funnel-shaped portion divided into two portions at a front end portion of the fistula expander, two grip bars secured to both sides of the funnel-shaped portion respectively, and a spring portion secured to rear end portions of the grip bars, and wherein the right and left portions of the fistula expander are secured to each other via a hinge generally at a middle portion of said two grip bars such that the right and left portions of the fistula expander can be opened and closed from side to side.

A fifth aspect of the present invention is an instrument for re-insertion of a gastrostoma tube, wherein an outer surface of the searching bar and an inner periphery of a lower end of the funnel-shaped portion of the fistula expander can slide freely up and down while they are kept in intimate contact with each other.

A sixth aspect of the present invention is an instrument for re-insertion of a gastrostoma tube as described above, wherein a lens aperture is defined in a side hole near a front end portion of the searching bar, and wherein the lens is connected to a light source and an image-receiving device via optical fibers which are passed through a lumen of the searching bar so that an inside of a stomach can be confirmed by images.

A seventh aspect of the present invention is an instrument for re-insertion of a gastrostoma tube as described above, wherein a lens aperture is defined in a leading edge of the searching bar, and wherein the lens at the leading edge is connected to a light source and image-receiving device via optical fibers which are passed through a lumen of the searching bar so that a state in close proximity to the front end of the searching bar can be seen by images.

An eighth aspect of the present invention is an instrument for re-insertion of a gastrostoma tube as described above, wherein lens apertures are defined in a leading edge of the searching bar and in a side surface near a front end of the searching bar, and wherein the respective lenses are connected to a light source and an image-receiving device via optical fibers which are passed through a lumen of the searching bar so that images in close proximity to the front end of the searching bar or images seen from the side surface of the front end can be optionally selected.

### BRIEF DESCRIPTION OF THE DRAWINGS

The present invention will be described herein below with reference to various figures, to which the scope of the present invention is not limited, in which:

FIG. 1 is a perspective view showing a searching bar of an instrument for re-insertion of a gastrostoma tube according to the present invention;

FIG. 2 is a partially enlarged view of a cross section of a front end portion in FIG. 1;

FIG. 3 is a perspective view showing a fistula expander of an instrument for re-insertion of a gastrostoma tube according to the present invention;

FIG. 4 is a sectional view taken along the line A-A of FIG. 3;

FIGS. 5 a and b are plan views showing a condition in which a fistula expander of FIG. 3 is opened and closed;

FIGS. 6 a-d are illustrations showing a way of using a searching bar of the instrument for re-insertion of a gastrostoma tube;

FIGS. 7 a-f are illustrations showing a way of using the instrument for re-insertion of a gastrostoma tube;

FIG. 8 is an illustration showing a condition in which a gastrostoma tube is used in a normal fistula;

FIG. 9 is a perspective view showing a searching bar of the instrument for re-insertion of a gastrostoma tube in the embodiment 2;

FIG. 10 is a partially enlarged view of a cross section of a front end portion in FIG. 9;

FIG. 11 is a perspective view showing a searching bar of the instrument for re-insertion of a gastrostoma tube in the embodiment 3;

FIG. 12 is a partially enlarged view of a cross section of a front end portion in FIG. 11;

FIG. 13 is a perspective view showing a searching bar of the instrument for re-insertion of a gastrostoma tube in the embodiment 4; and

FIG. 14 is a partially enlarged view of a cross section of a front end portion in FIG. 13.

### DESCRIPTION OF THE PREFERRED EMBODIMENTS

### EXAMPLE 1

FIG. 1 is a perspective view showing a searching bar 1 which constitutes an instrument for re-insertion of a gastrostoma tube according to the present invention. This searching bar 1 is a tubular body having an outer diameter of approximately 3 to 4 mm and the outline of the cross section of the tubular body is a smooth circle. As a result, it can slide smoothly in case of being combined with a lower portion of a funnel-shaped portion of a fistula expander while they are kept in intimate contact with each other.

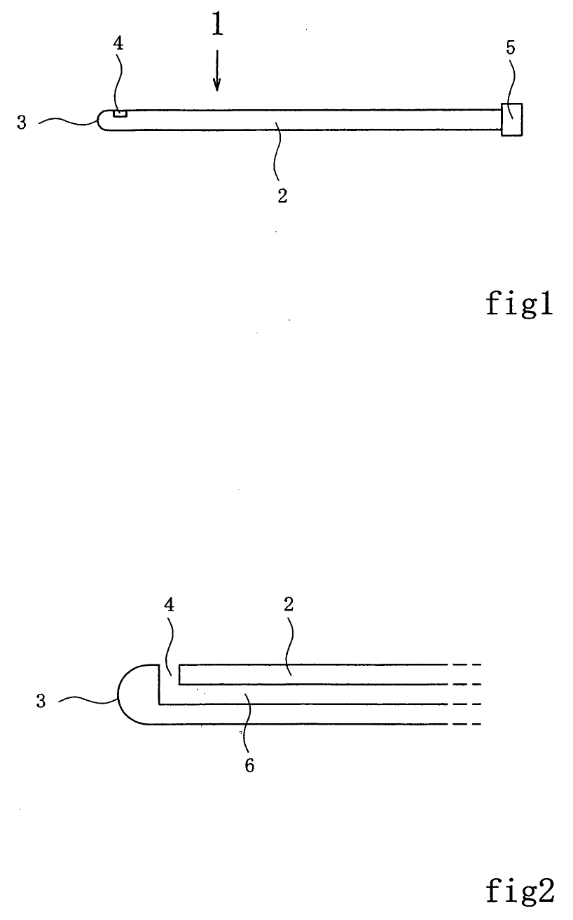

In addition, as shown in FIG. 2 which is a enlarged view of a cross section of a front end portion, the shape of the front end portion of the bar main body 2 is a hemispherical body 3 for prevention of hurts on an abdominal wall and a stomach wall after the gastrostoma tube is pulled out. Moreover, a side hole 4 is defined in a side surface of the neighborhood of the front end portion so that the side hole 4 can leads to the inside of the tube, and a connecting portion 5 is provided at a rear end of the bar so that a syringe (not shown) can be connected to the connecting portion 5.

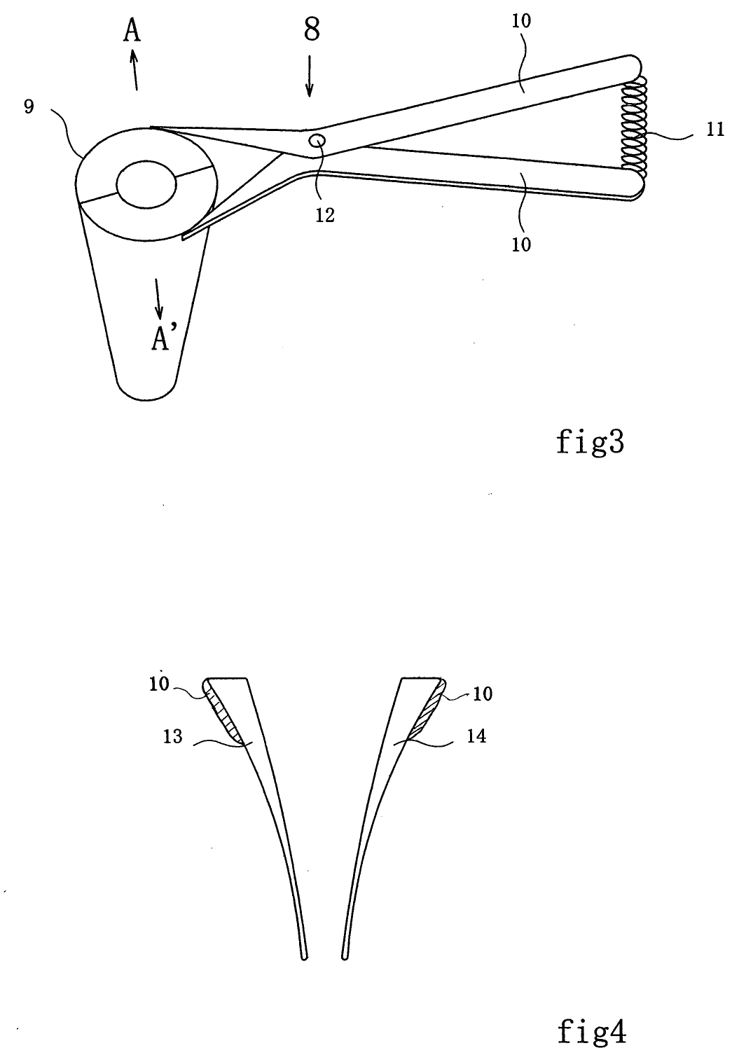

FIG. 3 is a perspective view showing a fistula-expander 8 which constitutes an instrument for re-insertion of a gastrostoma tube according to the present invention. The fistula expander 8 comprises a funnel-shaped portion 9 divided into two portions at a front end portion of fistula expander 8, two grip bars 10 secured to both sides of the funnel-shaped portion 9 respectively, and a spring portion 11 secured to rear end portions of the grip bars, and wherein the right and left portions of the fistula expander are secured to each other via a hinge 12 generally at a middle portion of the two grip bars such that the right and left portions of the fistula expander can be opened and closed from side to side.

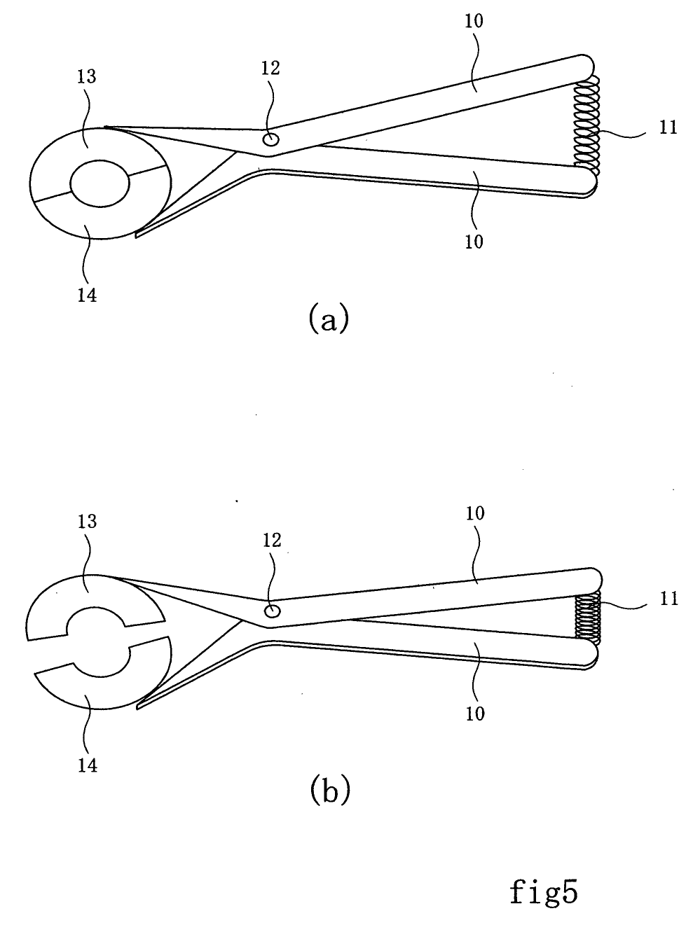

The above-mentioned funnel-shaped portion 9, which is divided into right and left funnel bodies as shown in FIG. 4, has a tapered shape in the direction of a front end of the funnel-shaped portion 9, wherein an inner diameter of the front end portion of the funnel-shaped portion 9 is designed to be slightly larger than the outer diameter of the above-mentioned searching bar.

The above-mentioned fistula expander 8 is designed such that, as shown in FIGS. 5 a and b, when gripping the grip bars 10, the spring portion attached to rear end portions of the grip bars is compressed and contracted, thereby allowing the right and left funnel bodies 14, 13 secured to front ends of the grip bars 10 to be opened, and then, the searching bar can be attached in such a way that the outside of the searching bar is pinched between the right and left funnel bodies 14, 13.

The instrument for re-insertion of a gastrostoma tube according to the present invention is a medical instrument used as a combination of the above-mentioned searching bar 1 and the above-mentioned fistula expander 8, which is used when exchanging for a new gastrostoma tube.

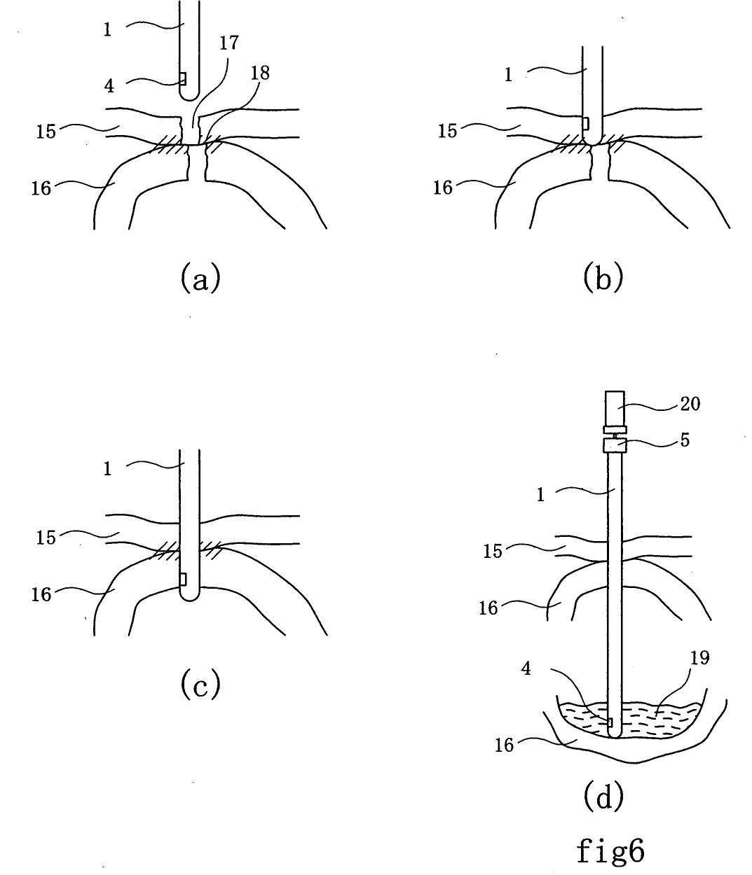

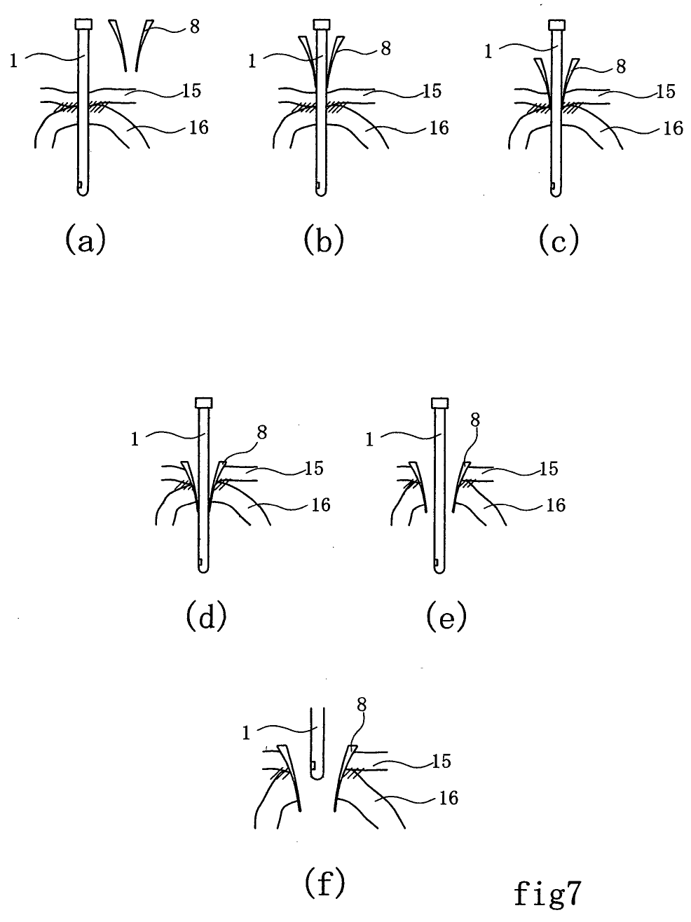

A way of using this instrument for re-insertion of a gastrostoma tube is shown in FIGS. 6 and 7. Firstly, after a gastrostoma tube (not shown) is pulled out, a front end 3 of the searching bar is applied on fistulae 17 of an abdominal wall 15 and a stomach wall 16, and is pressed gradually from the abdominal wall toward the stomach wall (FIG. 6a).

After passing through the abdominal wall 15, positions of the fistulae 17 may not be aligned with each other near an adhesion portion 18 between the abdominal wall 15 and the stomach wall 16 due to contraction of the fistulae 17 (FIG. 6 b). Therefore, while a hemispherical body which is a front end portion of the searching bar is pushed slowly, the location of the fistula of the stomach wall is confirmed, and thereafter, the front end of the searching bar is pressed further into the inside of the stomach (FIG. 6 c).

In order to confirm whether the instrument could be inserted into the inside of the stomach or not, a syringe 20 is attached to the connecting portion 5 at the rear end portion of the searching bar main body 2 and a gastric juice 19 in the stomach is aspirated from a side hole 4 in the neighborhood of the front end of the searching bar main body 2 for confirmation. In case that the juice is aspirated into a syringe, it is indicated that the front end of the searching bar reached the inside of the stomach (FIG. 6 d).

After the aspiration of the gastric juice from the inside of the stomach is confirmed, a fistula expander 8 is attached to the searching bar main body 2 in such a way as shown in FIG. 7. Firstly, the fistula expander 8 is moved to the neighborhood of a searching bar 1 inserted into a stomach (FIG. 7 a), and then, right and left funnel portions 14, 13 of the fistula expander 8 are opened so as to pinch and support the outside of the searching bar main body 2 (FIG. 7 b).

V The front ends of the right and left funnel portions 14 and 13 have such a structure that they can slide up and down while they are kept in intimate contact with the outer surface of the searching bar main body 2. Therefore, as the funnel portions 14, 13 are moved down gradually, the front ends of the funnel portions 14, 13 pass through the fistulae 17 of the abdominal wall 15, the adhesion 18 and the stomach wall 16, and is inserted through the inside of the stomach (FIGS. 7 c and d).

After confirming that the front ends of the funnel portions 14 and 13 are inserted through the inside of the stomach, the right and left funnel portions 14 and 13 are opened by gripping the grip bars of the fistula expander 8 (FIG. 7 e). Then, while maintaining the condition where they are opened, the searching bar is pulled out (FIG. 7 f), and a new gastrostoma tube is inserted for replacement and exchange.

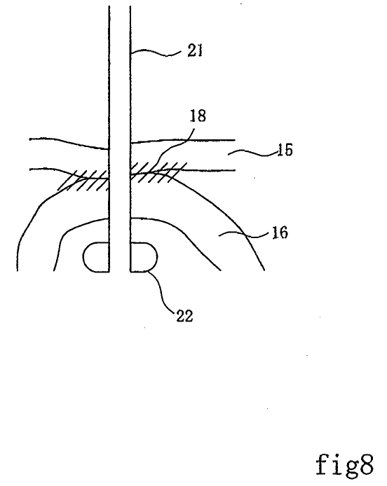

After the new gastrostoma tube is inserted through the inside of a stomach, the above-mentioned right and left funnel portions 14 and 13 of the fistula expander 8 are closed and gradually pulled upward reversely. The fistulae of the stomach wall 16, the adhesion portion 18 and abdominal wall 15 contract and press and secure the new gastrostoma tube 21 having a removal prevention balloon 22 (FIG. 8).

Although an instrument for re-insertion of a gastrostoma tube of the present invention is a medical instrument having a simple structure combining a searching bar 1 and a fistula expander 8 as stated above, it contributes greatly to treatments in a medical field because it can be easily manipulated.

### EXAMPLE 2

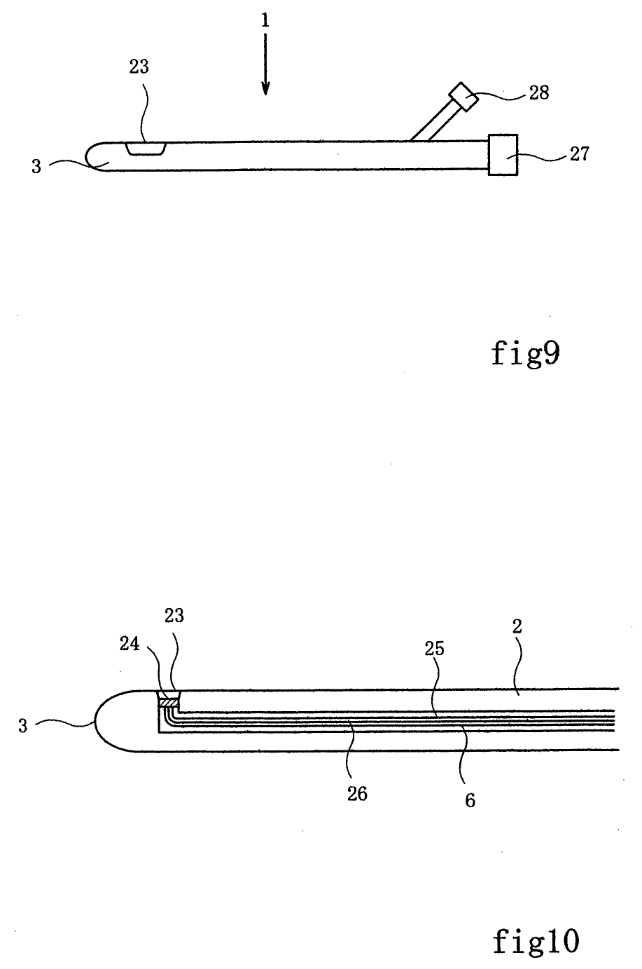

FIG. 9 is a perspective view showing a searching bar of an instrument for re-insertion of a gastrostoma tube used in another example of the present invention. The above-mentioned Example 1 has demonstrated that confirmation of the front end of a searching bar being introduced into the inside of a stomach can be provided by attaching a syringe to the rear end of the searching bar and aspirating a gastric juice of the inside of the stomach. In the present example, a searching bar 1 which is a tubular body having an outer diameter of approximately 3 to 4 mm is combined with a lower end of the funnel portion of the fistula expander so that the searching bar can slide smoothly while they are kept in intimate contact with each other in a manner similar to the example 1.

As shown in FIG. 10 which is a partially enlarged view of a cross section of a front end portion, the shape of the front end portion of the bar main body 2 is a hemispherical body 3 which prevents the front end portion from hurting an abdominal wall and a stomach wall after the gastrostoma tube is pulled out. In addition, a lens aperture 23 is defined in a side surface of the neighborhood of the front end portion, a lens 24 is attached to the inside of the lens aperture, and leads to optical fibers 25, 26 disposed in a tube inside 6, and their rear ends are connected to a light source 27 and an image-receiving device 28, respectively.

In this case, light emitted from the light source 27 is transferred through the fiber 26, and expanded and radiated outwardly from the lens aperture. As a result, with the image-receiving device 28, the condition outside the lens in the enlarged part illuminated by the light can be visually identified through the optical fiber 25 as well as photographs thereof can be taken when necessary.

### EXAMPLE 3

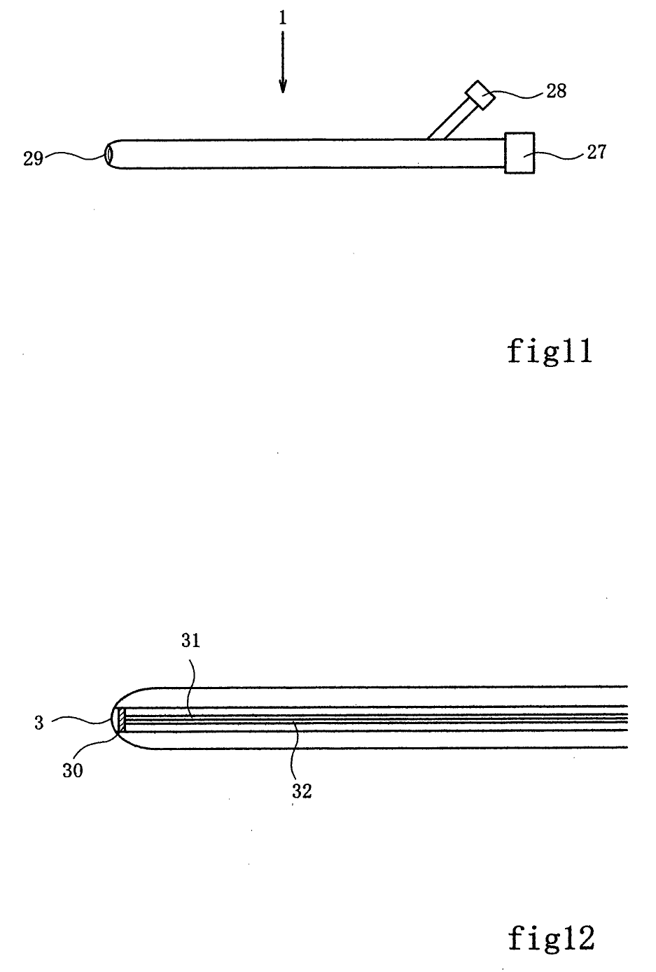

FIG. 11 is a perspective view showing a searching bar of an instrument for re-insertion of a gastrostoma tube used in another example of the present invention. In the present example, a searching bar 1 which is a tubular body having an outer diameter of approximately 3 to 4 mm is combined with a lower end of the funnel portion of the fistula expander so that the searching bar can slide smoothly while they are kept in intimate contact with each other in a manner similar to the example 1.

As shown in FIG. 12 which is a partially enlarged view of a cross section of a front end portion, the shape of the front end portion of the bar main body 2 is a hemispherical body 3 which prevents the front end portion from hurting an abdominal wall and a stomach wall after the gastrostoma tube is pulled out. In addition, a lens aperture 29 is defined in a front end of the neighborhood of the front end portion, a lens 30 is attached to the inside of the lens aperture, and leads to optical fibers 31, 32 disposed in the inside of the tube, and their rear ends are connected to a light source 27 and an image-receiving device 28, respectively.

In this case, light emitted from the light source 27 is transferred through the optical fiber, and expanded and radiated outwardly from the lens aperture 29 in the front end. As a result, with the image-receiving device 28, the condition outside the lens in the enlarged part illuminated by the light can be visually identified as well as photographs thereof can be taken when necessary.

### EXAMPLE 4

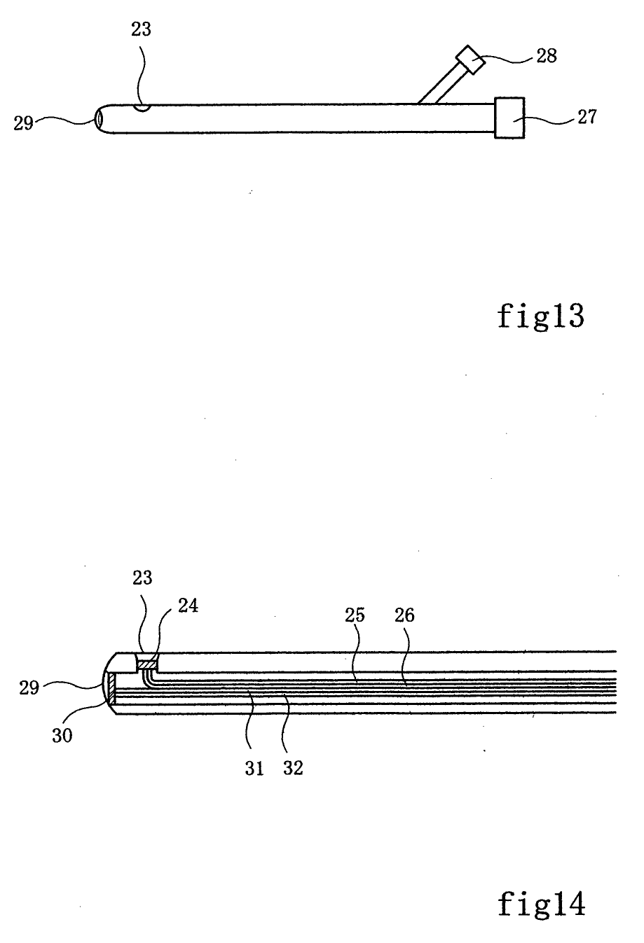

FIG. 13 is a perspective view showing a searching bar of the re-insertion instrument combining Example 2 and Example 3. In the present example, the searching bar 1 which is a tubular body having an outer diameter of approximately 3 to 4 mm as stated above, is combined with a lower end of the funnel portion of the fistula expander so that the searching bar 1 can slide smoothly while they are kept in intimate contact with each other in a manner similar to the example 1.

As shown in FIG. 12 which is a partially enlarged view of a cross section of a front end portion, the shape of the front end portion of the bar main body 2 is a hemispherical body 3 which prevents the front end portion from hurting an abdominal wall and a stomach wall after the gastrostoma tube is pulled out. In addition, a lens aperture 29 is defined in a front end of the neighborhood of the front end portion, a lens 24 is attached to the inside of the lens aperture, and leads to optical fibers 31, 32 disposed in the inside of the tube, and their rear ends are connected to a light source 26 and an image-receiving device 27, respectively. Besides, a lens aperture 23 is defined also in the neighborhood of the front end portion, a lens 24 is attached to the inside of the lens aperture, and leads to optical fibers 25, 26 disposed in the inside of the tube, and their rear ends are connected also to a light source 27 and an image-receiving device 28, respectively.

In this case, light emitted from the light source is transferred through the optical fiber, and expanded and radiated outwardly from either one or both of the lens aperture 29 in the front end and the lens aperture 23 in the neighborhood. As a result, with the image-receiving device, the condition outside the lens in the enlarged part illuminated by the light can be visually identified as well as photographs thereof can be taken when necessary.

Although the optical fibers shown in Examples 2 through 4 of the present invention have illustrated only two or four fibers, in fact, optical fibers which can be inserted through the inside of the tube will be connected, and plastic fibers are preferable to quartz fibers as a material of optical fiber.

An instrument for re-insertion of a gastrostoma tube of the present invention basically combines and uses a fistula expander and a searching bar. Therefore, when it is used, no difference in manipulation by an individual manipulator arises so that uniform treatments can be done.

Since the front end of the searching bar is a hemispherical body, it can pass through an abdominal wall portion of a contracting fistula, and then, the fistula of the stomach wall directly underneath the fistula of the abdominal wall portion can be searched out by the touch of the front end of the searching bar so that it can be inserted into the stomach. Therefore, intra-abdominal mis-insertion can be prevented.

Alternatively, the front end of the searching bar can be provided with an ultrasonic oscillator so as to provide a manner of searching a fistula of a stomach wall while observing the image by the ultrasonic oscillator.

In addition, it is possible to easily confirm that the front end of the searching bar itself is certainly inserted into a stomach by allowing the rear end portion of the searching bar to be a connecting portion to a syringe and by aspirating a gastric juice in the stomach from a side hole in the neighborhood of the front end of the searching bar into a syringe.

After confirming that the front end of the searching bar has been certainly inserted into the stomach, a funnel-shaped portion divided into two portions at a front end of the fistula expander is opened and set on the searching bar, and the front end of the funnel-shaped portion is moved down along the searching bar gradually through an abdominal wall and a stomach wall, and into the inside of the stomach. At a prescribed position, the fistula is expanded by opening the front end of the funnel-shaped portion by gripping two grip bars of the fistula expander so that a new gastrostoma tube can be inserted.

With a radiated light via the lens aperture defined in the front end portion and/or in the neighborhood of the front end portion of searching bar, which is transferred from a light source through optical fibers, it is possible to visually identify the inside of the stomach which is an outside of the lens aperture so that a new gastrostoma tube can be certainly inserted.

In addition, it becomes also possible to take photographs of an abdominal wall and a stomach-adhesion by means of the above-stated image-receiving device. Storing such photographs of the conditions of an abdominal wall, a stomach-adhesion, etc., can help management of a safe medical treatment.

1. A device for re-insertion of a gastrostoma tube comprising a combination of a searching bar and a fistula expander.

2. A device for re-insertion of a gastrostoma tube according to claim 1, wherein a main body of the searching bar is a tubular body, a front end of the searching bar presents a hemispherical shape, and a sidehole is defined near the front end.

3. A device for re-insertion of a gastrostoma tube according to claim 1 or 2, wherein a main body of the searching bar is a tubular body, and a connecting portion which can be connected to a syringe is provided at a rear end portion of the searching bar.

4. A device for re-insertion of a gastrostoma tube according to claim 1, wherein the fistula expander comprises a funnel-shaped portion divided into two portions at a front end portion of the fistula expander, two grip bars secured to both sides of the funnel-shaped portion respectively, and a spring portion secured to rear end portions of the grip bars, and wherein the right and left portions of the fistula expander are secured to each other via a hinge generally at a middle portion of said two grip bars such that the right and left portions of the fistula expander can be opened and closed from side to side.

5. A device for re-insertion of a gastrostoma tube according to claim 1 or 2, wherein an outer surface of the searching bar and an inner periphery of a lower end of the funnel-shaped portion of the fistula expander can slide freely up and down while they are kept in intimate contact with each other.

6. A device for re-insertion of a gastrostoma tube according to claim 1, wherein a lens aperture is defined in a side hole near a front end portion of the searching bar, and wherein the lens is connected to a light source and an image-receiving device via optical fibers which are passed through a lumen of the searching bar so that an inside of a stomach can be confirmed by images.

7. A device for re-insertion of a gastrostoma tube according to claim 1, wherein a lens aperture is defined in a leading edge of the searching bar, and wherein the lens at the leading edge is connected to a light source and an image-receiving device via optical fibers which are passed through a lumen of the searching bar so that a state in close proximity to the front end of the searching bar can be seen by images.

8. A device for re-insertion of a gastrostoma tube according to claim 1, wherein lens apertures are defined in a leading edge of the searching bar and in a side surface near a front end of the searching bar, and wherein the respective lenses are connected to a light source and an image-receiving device via optical fibers which are passed through a lumen of the searching bar so that images in close proximity to the front end of the searching bar or images seen from the side surface of the front end can be optionally selected.
